(19) United States
(12) Patent Application Publication
(10) Pub. No.: US 2006/0100508 A1
(43) Pub. Date: May 11, 2006

(54) Method and apparatus for expert system to track and manipulate patients

(76) Inventor: Matthew M. Morrison, Cordova, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 10/985,108

(22) Filed: Nov. 10, 2004

(51) Int. Cl. A61B 5/05

(52) U.S. Cl. 600/426; 600/414

(57) ABSTRACT

A system and method to track and manipulate the bony anatomy of a patient. A plurality of fiducials is placed on the bony anatomy of a patient. The relative spatial relationships of the fiducials are scanned and a signal generated before, during and after a surgical procedure. The patient is manipulated according to predetermined relative spatial relationships to provide a successful clinical outcome. The fiducials may be LED devices capable of optical observation or signal-emitting devices capable of spatial relationships in three dimensions. An expert system allows comparison of the relative spatial relationships to historical, healthcare facility and Internet databases to plan and determine clinical success of a patient procedure.

### FIELD OF THE INVENTION

The invention relates to a patient manipulation and location tracking system.

### BACKGROUND OF THE INVENTION

In the medical field, and more specifically in the field of surgery, there exists a strong need for real time accurate illustrations of a patient's anatomy, and especially the bony anatomy. This becomes an issue in, for example, spinal surgery and hip replacement. Heretofore real time location of the bony anatomy has been done through the use of x-rays in a computer aided tomography (CAT) scan. This provides an image observable by the surgeon while an operation is in progress to determine whether the bony parts of the anatomy are being manipulated into correct relationship for a successful clinical outcome. Furthermore, images collected through x-rays can be observed prior to surgery to map out a procedure and planned outcome.

Another approach to real time determination of patient anatomy is found in the Flouronav system originating with SMT Corporation of Louisville, CO. In this system, an x-ray image of the patient is acquired, stored and portrayed in three-dimensional fashion. Fiducials in the form of fixed markers showing up on the x-rays are included to coordinate or register the images in real time.

Still another approach is an interventional MRI (Magnetic Resonance System) wherein a patient is positioned within the MRI, images acquired and then the patient removed to permit surgical intervention. Sequential insertion and removal from the MRI allows the surgeon to determine location of a patient's anatomy.

The above approaches, while generally giving an indication of the relative location of the bony anatomy and other patient members, suffer from a number of deficiencies. The first deficiency is the complexity and expense of the scanning system, both in terms of bulk and in cost. With systems costing over several million dollars, it is not practical to deploy them in large numbers in surgical suites. The second drawback is the addition of complicated imaging procedures onto an already demanding procedure.

A further deficiency of current procedures is that the experienced surgeon needs to mentally visualize expected locations of the bony anatomy to achieve a successful clinical outcome. This capability can be achieved only after years of practice in performing similar types of surgery.

In addition, the surgeon has expected clinical outcomes based on results of many years of surgery. This slows the rise of a skill level by basing it on actual surgeries performed. There have been many proposals for systems of the x-ray type to incorporate databases to allow for accumulated knowledge and information to the trade. However, systems of this type use various forms of x-rays or MRI so that continual irradiation is expensive and adds complication.

Thus a need exists in the art to achieve a real time spatial relationship of a patient's anatomy and the ability to incorporate in this observation experience-based knowledge of others in the field.

### SUMMARY

In one aspect of the invention a method for determining time spaced spatial relationships on a patient comprises a number of steps. These include placing a plurality of fiducials on the patient and scanning and generating a signal reflecting the relative spatial relationship of the fiducials. The signals are stored and subsequently the fiducials are scanned and generate a signal which reflects the then-current relative spatial relationship of the fiducials.

In another aspect of the invention, the invention relates to apparatus for determining time spaced spatial relationships on a patient. The apparatus comprises a plurality of fiducials placed on the patient. A device for generating a signal as a function of the relative spatial relationship of the fiducials is provided and a device receives the signals and stores the relative spatial relationships, the device being capable of time spaced storage of the relative spatial relationships.

In yet another aspect of the invention, a computer-assisted method of patient treatment is provided and comprises the steps of: providing a resident historical database of patient treatment pathways including patient conditions; receiving an online link to selected patient treatment pathways data; receiving an input representing real time patient condition; receiving consultative data on patient treatment pathways and condition; and determining from the historical data, online data, real time input and consultative data a specific treatment pathway for the patient.

In yet another aspect of the invention, the treatment pathway is altered periodically based on real time condition.

In yet another aspect of the invention, the method has the further step of placing a plurality of fiducials on the patient and scanning and generating a signal reflecting the relative spatial relationship of the fiducials to determine real time patient condition.

### A BRIEF DESCRIPTION OF THE DRAWINGS

FIG. 1 is a schematic drawing of a patient with a system for spatial relationships, which embodies the present invention.

FIG. 2 is a perspective view of a fiducial that is incorporated in the system shown in FIG. 1.

FIG. 3 is an enlarged view of several vertebrae with attached fiducials, showing an exemplary type of movement.

FIG. 4 is a schematic drawing showing the expert system used in conjunction with the patient treatment apparatus of FIG. 1.

FIG. 5a is a process flow diagram for the system of FIG. 1 and FIG. 2 in a pre-treatment condition.

FIG. 5b is a process flow diagram for the system of FIG. 1 and FIG. 2 showing the steps conducted as the patient treatment is undertaken.

### DESCRIPTION OF THE SELECTED EMBODIMENT

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described processes, systems or devices, and any further applications of the principles of the invention as described herein, are contemplated as would normally occur to one skilled in the art to which the invention relates.

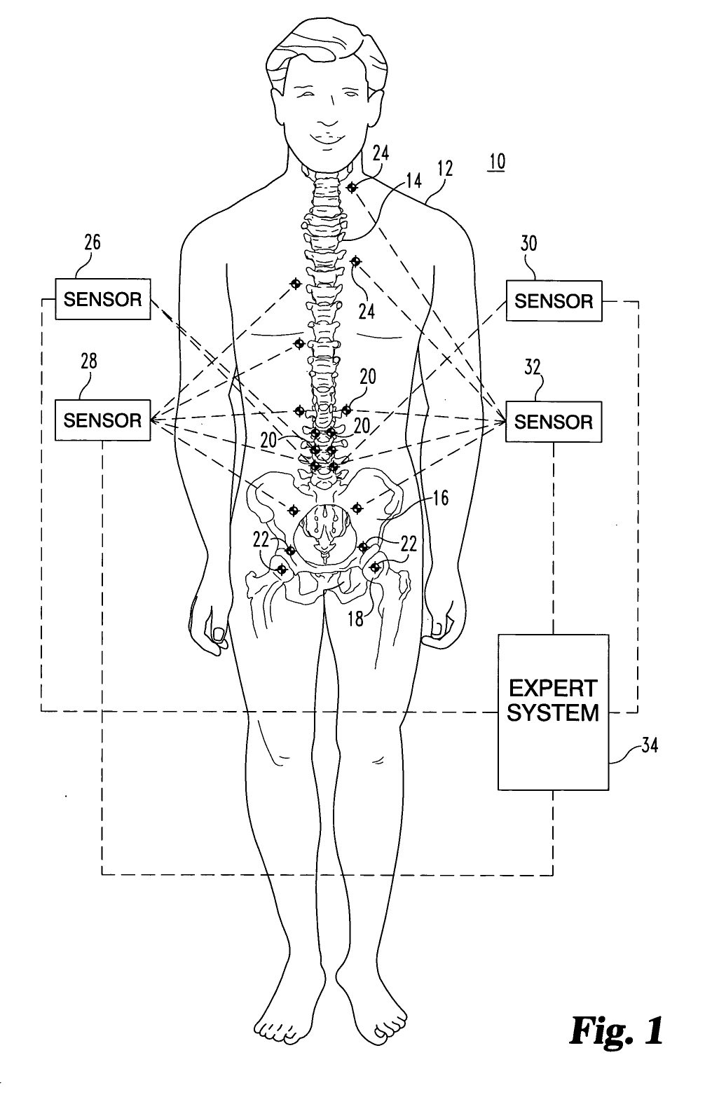

FIG. 1 illustrates a patient 12 with an expert patient treatment system identified by the reference character 10. The patient 12 is shown with the spine 14 exposed as well as the pelvic area 16 including hip joints 18. In accordance with the present invention, a plurality of fiducials 20 are placed on the spine. A further plurality of fiducials 22 are placed around the hip joints and additional fiducials 24 may be employed in other areas of a patient. In the literature a fiducial is defined as a “way point” or “reference point.” In medical technology, fiducials are usually elements affixed to portions of a patient's body, especially the bony anatomy, that are particularly highlighted in an x-ray so that succeeding scans or pictures may have registration with one another.

In accordance with the present invention, the fiducials 20, 22 and 24 are of a special form enabling them to be mapped in three-dimensional format relative to one another. The fiducial in accordance with the present invention may take a number of forms but two main forms are shown for illustrative purposes. A first form contemplates the fiducials as being capable of visual observation such as LEDs for determination of relative position. The second form involves an active fiducial which generates a signal capable of triangulation by means of sensors 26, 28, 30 and 32 that generate signals showing the relative spatial relationships of the fiducials and transmit those signals to an expert system 34. The principal of the invention is that the fiducials are placed at strategic locations on a patient, for example the bony anatomy, so that relative spatial relationships between the fiducials can be determined to map out treatment pathways, adjust treatment pathways and to determine clinical success. All this is done without the need for expensive x-rays in their various forms. By not having this added complication, determination of relative spatial relationships can be made more frequently and even after any patient procedures.

Whether fiducials are LED's or active sensors, their relative spatial relationships are determined by the sensors 26-32. These sensors determine, optically or by means of a signal emanating from the fiducials, their relative position in three dimensions. Such position is typically done by triangulation on the particular signals. Triangulation can be done either by lateration, i.e. determining distance measurements to the fiducials from the sensors, or by angulation. Angulation involves determining angles between the sensors and the fiducials and computing location of the fiducials based on the fixed dimensions between the sensors. Such approaches to determining three-dimensional measurements are well known in the art and will not be repeated in order to simplify and focus on an understanding of the present invention. The sensors 26-32 would be optical sensors in case the fiducials are LED's and will be, for example, radio direction sensors when fiducials 20-24 are RF (radio frequency) transmitters.

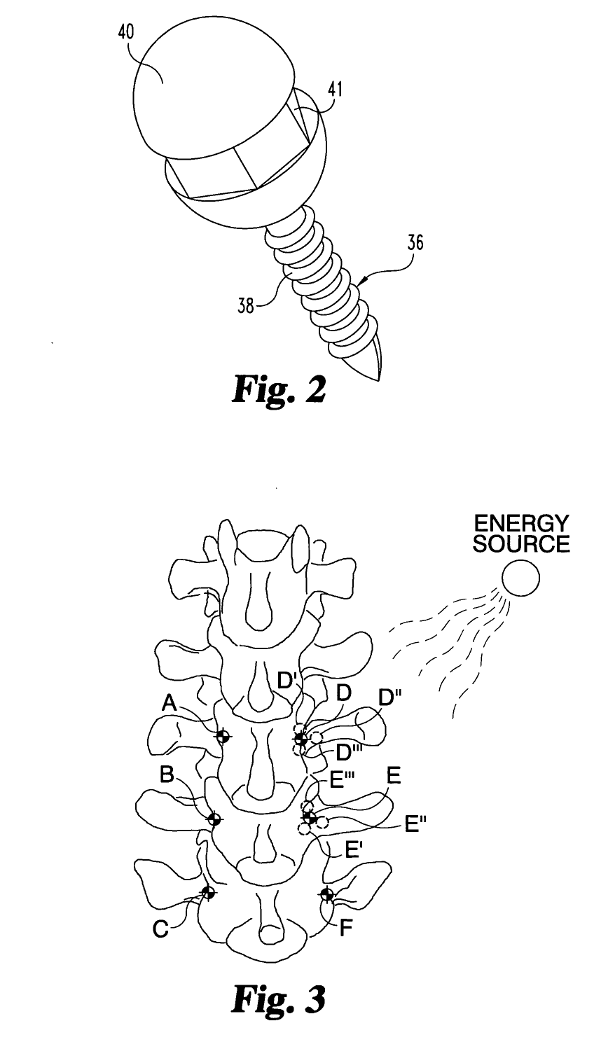

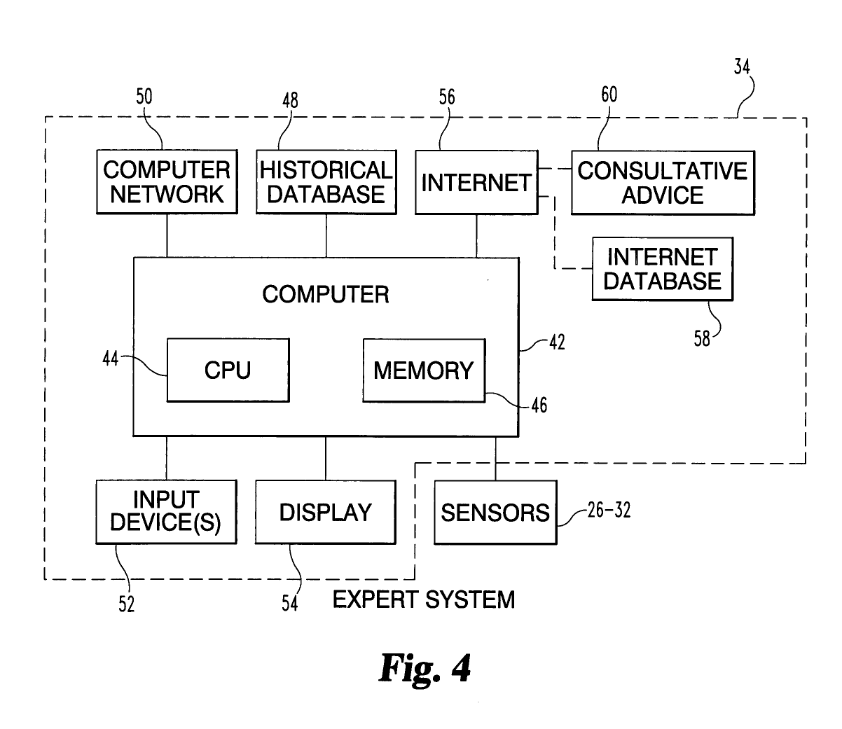

FIG. 2 shows an example of a fiducial employed in the patient treatment system of FIG. 1. The fiducials 20, 22, 24 comprise a base 36 with threads 38 for appropriate fastening to the bony anatomy of a patient. A sensor housing 40 contains circuitry and electronics for generating an electrical signal in response to being subjected to an energy field, usually of radio frequency signals. A hex section 41 allows an appropriate tool (not shown) to apply a torque to the fiducial to screw it into the bone or remove it. The circuitry within sensor housing 40 generates a signal, which is then captured by sensors 26-32 and triangulated to produce a signal reflecting the relative positions of the fiducials.

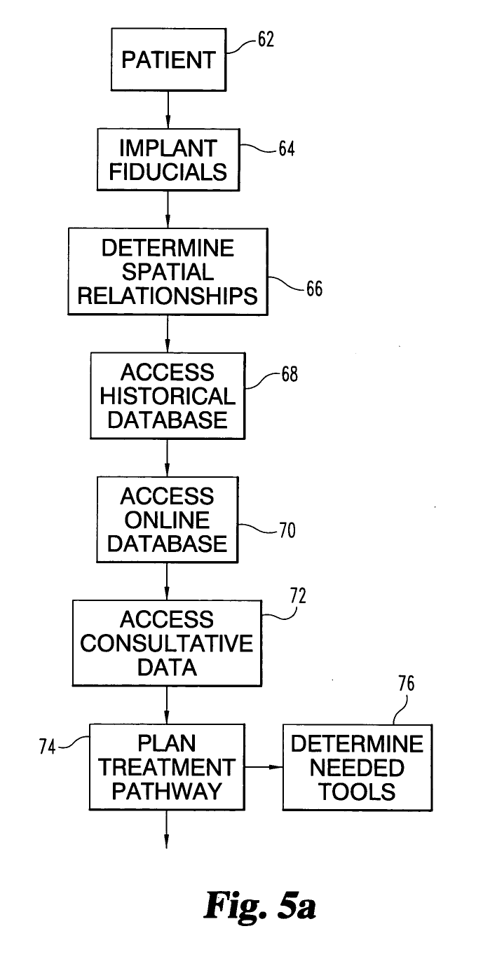

FIG. 4 shows an example of how the relationship between fiducials 20, fastened to the vertebrae of the spine, may be used to indicate the lateral, flexion and extension of the spine. In this example, the fiducial location will be identified by letters of the alphabet shown in FIG. 4.

In the treatment of conditions such as Scoliosis, the flexibility of the spine is determined for purposes of taking appropriate corrective action. There are a number of ranges of motion of the lumbar spine that can be sensed by the fiducials 20. These include flexion which determines the extent to which a patient can bend forward, lateral flexion which determines how a patient can bend to the side and then extension, meaning the increase in distance between adjacent vertebrae. All of these ranges of movement can be determined by appropriate positioning of the fiducials 20. However, for purposes of explanation, the lateral flexion will be described with reference to FIG . 4. The center lines represented by D and E indicate location of the vertebrae at rest. There is a fixed relationship between \overline{AD} and \overline{BE}. When a patient is asked to bend the spine to the side without rotating or tilting the pelvis, fiducials D′ and E will be displaced relative to one another. The phantom center line indicated by D′ signifies movement in a vertical direction as viewed in FIG. 4, D″ signifies movement to the right and D′″ signifies movement towards the bottom of the figure. Likewise, phantom center line E′″ signifies movement in a vertical direction in FIG. 4, E″ signifies movement to the right and E′ signifies movement to the lower part of FIG. 4. In measuring the movement during bending of the lumbar region of the spine, the relative lateral flexion is indicated by the vectors \overline{DD}″ and \overline{EE}″ or \overline{DE}″ and \overline{ED}″. The relative magnitude of these measurements can be used to determine in an accurate way the lateral flexion of the lumbar region of the spine. This gives a far more accurate determination of the condition of the spine than previous practices of using indicators external to the body to indicate the amount of flexion. Furthermore, the use of the fiducials in accordance with this invention provide a far more cost effective and straight-forward way to determine relative positions.

Appropriate placement and determination of displacement can be employed for flexion and for extension. The advantages of having fiducials with the capability to emit signals is that the fiducials may be retained in a patient after initial placement for continuing interrogation and determination of displacement, employing the principal that most of the information desired to be secured is captured by selective determination of critical points rather than x-ray scans of the entire anatomy of a patient.

The advantage of the fiducials is that they may be inserted in place in a patient before surgery and their relative location determined to provide a patient treatment plan. Since fiducials are relatively compact, they may be left within the patient during the surgery and after the surgery to access the clinical success of the procedure. It is also possible that the fiducials may be biodegradable so as to avoid the need for surgical extraction of the fiducials when surgery is completed and clinical success is verified. The advantage of the fiducials staying in a patient after a procedure is that it provides an effective and straightforward method for determining whether relationships planned for and manipulated in the procedure continue to be in the position intended. For example, in a hip replacement procedure the sensors could determine relative movement when it should not be occurring.

The relative position of the fiducials 20 is sensed by sensors 26-32. The signals from the sensors 26-32 are fed to the expert system 34 shown in block diagram in FIG. 4. Expert system 34 comprises a computer 42 having a CPU 44 and memory 46. The computer receives inputs from a plurality of devices and systems including sensors 26-32. A resident historical database 48 includes spatial relationships and other data indicative of patient treatment plans. A computer network 50 interconnects the computer 42 to other areas of a healthcare facility's network. Additional input devices 52 are in the form of barcode readers or other devices and systems to detect and identify various tools needed for patient procedure. A display 54 indicates the spatial relationship of the data fed to the computer by sensors 26-32. An additional interconnection to the Internet 56 through computer 42 provides access to an Internet database 58 and a consultative advice database 60.

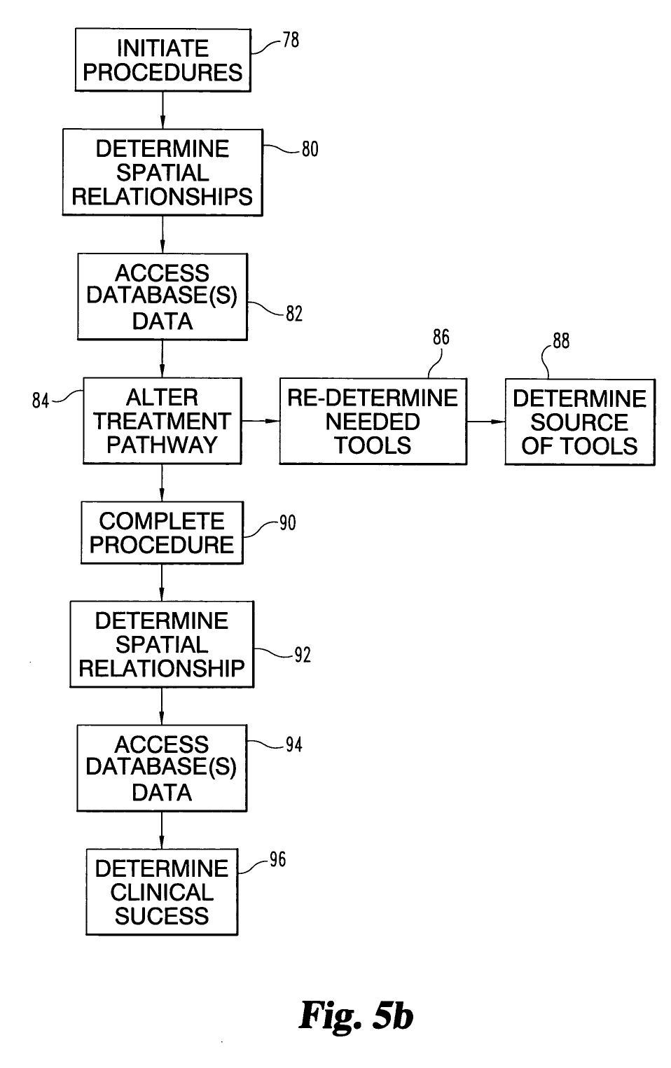

The devices and systems set forth in FIG. 4 are used to implement an expert system procedure set forth in FIG. 5a and FIG. 5b. In FIG. 5a, a patient 62 has fiducials implanted at 64 in locations appropriate for the procedures involved. For example, if the procedure involves correction of a spinal disorder like Scoliosis, the fiducials are implanted in the spine as shown for fiducials 20. Once the fiducials 20 are in place, the fiducials 20 are interrogated to determine the spatial relationships of the fiducials at 66. The historical database is accessed at 68 to help plan a patient treatment pathway based on relative fiducial positions in prior clinical experiences. In addition to the historical database 48, access to additional patient treatment may be provided by accessing the Internet database 70. In addition, during this period, access to consultative advice and data may be provided at 72. The spatial relationships, historical database, Internet database and the consultative data are used to plan a treatment pathway at 74 including a step of determining needed tools for the procedure at 76.

In FIG. 5b, the procedures are initiated at 78 and the spatial relationships are again determined at 80. The time spaced spatial relationships are then compared to the various databases shown in FIG. 4 at 82. Based on the access to the various databases, the treatment pathway may be altered at 84. This may involve the additional step of re-determining needed tools at 86 and also determining the source of the tools at 88. This may be provided by the input devices 52 shown in FIG. 4. For example, appropriate tools for a procedure may be bar-coded and identified through means of optical scanning. The tools and source of the tools may also be determined by other means. Based on the re-determination of the spatial relationships at 80 and any needed change in the required tools, the procedure is completed at 90 and the spatial relationships are again determined at 92. The additional step of accessing the database or databases at 94 allows a comparison with the planned patient treatment pathway fiducials position and actual to determine clinical success at 96.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed

1. A method for determining time spaced spatial relationships on a patient, said method comprising the steps of:
placing a plurality of fiducials on said patient,
scanning and generating a signal reflecting the relative spatial relationship of said fiducials,
storing said signals, and
subsequently scanning and generating a signal reflecting the then-current relative spatial relationship of said fiducials.

2. A method as claimed in claim 1 wherein the patient is a surgical patient and said step of scanning and generating a signal reflecting the relative spatial relationship of said fiducials is taken before surgery and said method further comprises the step of manipulating the patient to synchronize the spatial relationship of said fiducials with a predetermined spatial relationship.

3. A method as claimed in claim 1 wherein the spatial relationships of said fiducials for scanning and generating a signal is in three dimensions.

4. A method as claimed in claim 2 wherein said fiducials are scanned and a signal generated after a surgical operation.

5. A method as claimed in claim 4 wherein said fiducials are compared to a predetermined clinical success relative spatial relationship.

6. A method as claimed in claim 1 wherein said fiducials are placed on the bony anatomy of a patient.

7. A method as claimed in claim 1 wherein said fiducials are scanned and said signal generated through visual observation of said fiducials.

8. A method as claimed in claim 7 wherein said fiducials are LED emitters and said scanning and signal generating is done using a digital camera.

9. A method as claimed in claim 1 wherein said fiducials emit a signal and said scanning and signal generating is done using signals emanating from said fiducials.

10. Apparatus for determining time spaced relative spatial relationships on a patient, said apparatus comprising:
a plurality of fiducials placed on said patient,
a device for generating a signal as a function of the relative spatial relationship of said fiducials,
a device receiving said signals and storing said spatial relationships, said device being capable of time spaced storage of said spatial relationships.

11. Apparatus as claimed in claim 10 wherein said signal receiving device stores predetermined relative spatial relationships for said fiducials and compares the actual location with the predetermined locations.

12. Apparatus as claimed in claim 10 wherein said predetermined relative spatial relationships reflect a successful clinical outcome.

13. Apparatus as claimed in claim 10 wherein said device for generating a signal and the device for receiving said signal determines the spatial relationship in at least one of before, during and after a surgical procedure.

14. Apparatus as claimed in claim 12 wherein said device for generating a signal and the device for receiving said signal determine the spatial relationships before and during the surgical procedure.

15. Apparatus as claimed in claim 12 wherein said device for generating a signal and the device for receiving said signal determine the relative spatial relationships of said fiducials before, during and after the surgical procedure.

16. Apparatus as claimed in claim 10 wherein said fiducials are placed on the bony anatomy of said patient.

17. Apparatus as claimed in claim 10 wherein said patient is a surgical patient.

18. Apparatus as claimed in claim 10 wherein said fiducials are light emitting devices and said signal generating device generates a signal in response to the location of said LED devices.

19. Apparatus as claimed in claim 10 wherein said fiducials emit a signal and said signal-generating device is responsive to said signal for determining the spatial relationships of said fiducials.

20. Apparatus as claimed in claim 19 wherein said fiducials are responsive to presence in an energy field to generate a signal and wherein said signal-generating device applies energy to said fiducials to generate said signals.

21. Apparatus as claimed in claim 20 wherein said signal-generating device determines the position of said fiducials by triangulation to generate said signals.

22. A computer assisted method of patient treatment comprising the steps of:
providing a resident historical database of patient treatment pathways including patient conditions,
receiving an online link to selected patient treatment pathways data,
receiving an input representing real time patient condition, and
receiving consultative data on patient treatment pathways and condition, and
determining from the historical data, online data, real time input and consultative data a specific treatment pathway for said patient.

23. A method as claimed in claim 22 further comprising the step of adding real time patient conditions to said resident historical database.

24. A method as claimed in claim 22 comprising the further steps of periodically determining real time patient condition and determining from said data and real time condition an altered treatment path for said patient.

25. A method as claimed in claim 22 further comprising the step of filtering of consultative data.

26. A method as claimed in claim 25 wherein said filtering step comprises a qualitative measure on said consultative data

27. A method as claimed in claim 22 wherein said resident historical database of patient treatment pathways are predicted treatment pathways.

28. A method as claimed in claim 27 wherein said treatment pathway is altered periodically based on real time patient condition.

29. A method as claimed in claim 27 wherein said treatment pathways are generated from a probabilistic treatment pathway.

30. A method as claimed in claim 28 further comprising the step of providing tool identification for treatment of said patient.

31. A method as claimed in claim 30 wherein indicia are provided on said tools and said method further comprises the step of indicating the proper tool based on indicia on the tools.

32. A method as claimed in claim 30 wherein said step of identifying required tools is performed through an online link.

33. A method as claimed in claim 32 wherein said step of determining patient condition comprises placing a plurality of fiducials on said patient and scanning and generating a signal reflecting the relative spatial relationship of said fiducials.
